(19) United States
(12) Patent Application Publication
(10) Pub. No.: US 2025/0331819 A1
(43) Pub. Date: Oct. 30, 2025

(54) ULTRASONIC PROBE

(71) Applicant: GE Precision Healthcare LLC, Waukesha, WI (US)

(72) Inventors: Kotaro Takeda, Hino (JP); Rajeev Mandwal, Zipf (AT); Atsushi Tsujimoto, Hino (JP)

(21) Appl. No.: 19/190,497

(22) Filed: Apr. 25, 2025

(30) Foreign Application Priority Data
Apr. 25, 2024 (JP) 2024-071895

(51) Int. Cl. A61B 8/00

(52) U.S. Cl. CPC A61B 8/4444; A61B 8/4254; A61B 8/467; A61B 2560/0462; A61B 2562/0223

(57) ABSTRACT

An ultrasonic probe, including an ultrasonic transducer; a metal inner housing that is thermally connected to the ultrasonic transducer; and a magnetic flux line generator disposed at a position at least partially surrounded by the inner housing and having a first pole and a second pole, wherein the inner housing comprises a magnetic flux line-passing structure that allows passage of magnetic flux lines from the first pole back to the second pole.

### CROSS-REFERENCE TO RELATED APPLICATION

This application claim priority to Japanese Patent Application No. 2024-071895, which was file on Apr. 25, 2024 at the Japanese Patent Office. The entire contents of the above-listed application are incorporated by reference herein in their entirety.

### TECHNICAL FIELD

The present invention relates to an ultrasonic probe, and more particularly to an ultrasonic probe provided with a magnetic flux line-passing structure.

### BACKGROUND

When performing an ultrasonic examination, an operator can dispose an ultrasonic probe at any position on a scan target, orient the probe in any direction, perform imaging, and obtain a non-destructive/non-invasive ultrasonic image.

When performing such an ultrasonic examination, a known method is, for example, to dispose a transmitter that generates a magnetic field outside of the ultrasonic probe and attach a magnetic sensor to the ultrasonic probe to detect the position and direction of the ultrasonic probe, for the purpose of linking with images from other modalities such as an X-ray CT (X-ray computed tomography) device and an MRI (magnetic resonance imaging) device (for example, patent document 1 and non-patent document 1).

In the method of detecting the position and orientation of the ultrasonic probe using the magnetic sensor, there is a problem in that the detection accuracy deteriorates when interference with the magnetic field from the transmitter is added and the magnetic field is distorted, or when the magnetic field strength is insufficient. In a magnetic sensor using a magnetic field as a signal, when there is a mass of a ferromagnetic material or a metal serving as a source of an eddy current around the magnetic sensor, the signal may be blocked and the magnetic sensor may not function.

On the other hand, the ultrasonic transducer included in the ultrasonic probe vibrates in response to an applied voltage and generates heat because it is a constituent element that generates ultrasonic waves. In order to dissipate heat generated by the ultrasonic transducer, the ultrasonic probe may be provided with a metal inner housing that is thermally connected to the ultrasonic transducer. Since the inner housing must have high thermal conductivity, it must be manufactured using a metal having high thermal conductivity, such as aluminum or copper.

For this reason, in the conventional art, the magnetic sensor is disposed outside of the inner housing so that the metal inner housing does not interfere with the magnetic field from the transmitter.

Disposing the magnetic sensor outside of the inner housing may cause one or more of the following problems: an increase in the size of the ultrasonic probe, a decrease in the ease of operation by an operator, time being required for hygiene management such as cleaning/sterilization, a possibility of shortening the life of the ultrasonic probe due to an increase in the number of positions where stress concentration occurs, and difficulty in providing an aesthetic design.

### SUMMARY

In a first aspect of the present disclosure, an ultrasonic probe is provided. The ultrasonic probe is provided with an ultrasonic transducer, a metal inner housing that is thermally connected to the ultrasonic transducer, and a magnetic flux line generator disposed at a position at least partially surrounded by the inner housing and having a first pole and a second pole. The inner housing is provided with a magnetic flux line-passing structure that allows passage of magnetic flux lines from the first pole back to the second pole.

In a second aspect of the present disclosure, an ultrasonic diagnostic device provided with an ultrasonic probe is provided. The ultrasonic probe is provided with an ultrasonic transducer, a metal inner housing that is thermally connected to the ultrasonic transducer, and a magnetic sensor disposed at a position at least partially surrounded by the inner housing. The inner housing is provided with a magnetic flux line-passing structure that allows passage of magnetic flux lines for detecting a magnetic field generated by a transmitter disposed outside of the ultrasonic probe.

In a third aspect of the present disclosure, an ultrasonic diagnostic device provided with an ultrasonic probe is provided. The ultrasonic probe is provided with the features of the first aspect of the present disclosure or the second aspect of the present disclosure.

In a fourth aspect of the present disclosure, an ultrasonic diagnostic system provided with an ultrasonic diagnostic device and a transmitter that generates a magnetic field to be detected by a magnetic sensor is provided. The ultrasonic probe of the ultrasonic diagnostic device is provided with the features of the first aspect of the present disclosure or the second aspect of the present disclosure.

### BRIEF DESCRIPTION OF THE DRAWINGS

FIG. 1 A block diagram illustrating one example of a schematic configuration of an ultrasonic diagnostic system according to an embodiment of the present invention.

FIG. 2 A diagram illustrating an external structure and an internal structure of an ultrasonic probe.

FIG. 3 A diagram illustrating the internal structure of the ultrasonic probe.

FIG. 4 An exploded view illustrating main components of the ultrasonic probe.

FIG. 5 A diagram illustrating a chassis built into the ultrasonic probe.

FIG. 6A A diagram illustrating the disposal of solenoids and electronic components of a magnetic sensor.

FIG. 6B A diagram illustrating the disposal of the solenoids and the electronic components of the magnetic sensor.

FIG. 6C A diagram illustrating the disposal of the solenoids and the electronic components of the magnetic sensor.

FIG. 7A A diagram illustrating the disposal of the solenoids and metal components of the magnetic sensor.

FIG. 7B A diagram illustrating the disposal of the solenoids and the metal components of the magnetic sensor.

FIG. 7C A diagram illustrating the disposal of the solenoids and the metal components of the magnetic sensor.

FIG. 7D A diagram illustrating the disposal of the solenoids and the metal components of the magnetic sensor.

FIG. 8A A diagram illustrating an eddy current generated in a metal by magnetism.

FIG. 8B A diagram illustrating an eddy current generated in a metal by magnetism.

FIG. 9 A diagram illustrating the disposal of the solenoids and an inner housing of the magnetic sensor.

FIG. 10 A diagram illustrating the inner housing in a preferred embodiment.

### DETAILED DESCRIPTION

Embodiments of the present invention will be described below. Note that the invention claimed in the embodiments described herein is not limited. In particular, in the present disclosure, a medical ultrasonic diagnostic system is described as an example. However, the present invention may be applied to an ultrasonic examination system, an ultrasonic examination device, and an ultrasonic probe for the non-destructive examination of buildings, structures, various mechanical devices, and the like.

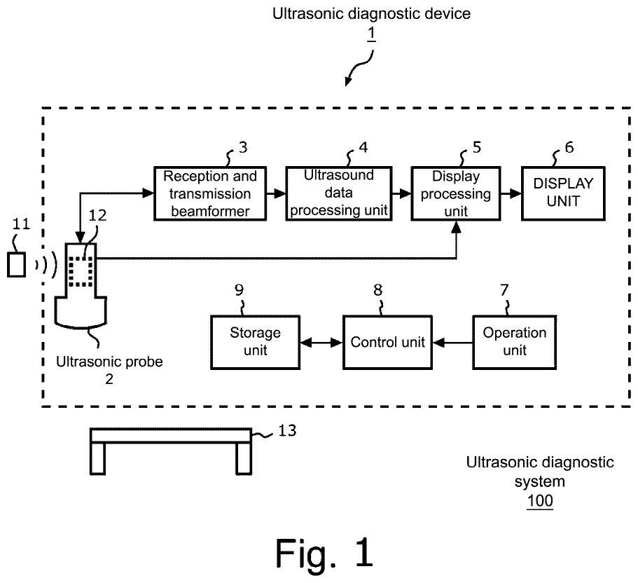

Embodiments of the present invention will be described hereinafter with reference to the drawings. The ultrasonic diagnostic system 100 illustrated in FIG. 1 includes the ultrasonic diagnostic device 1. The ultrasonic diagnostic device 1 is provided with an ultrasonic probe 2, a reception and transmission beamformer 3, an ultrasound data (echo data) processing unit 4, a display processing unit 5, a display unit 6, an operation unit 7, a control unit 8, and a storage unit 9. The ultrasonic diagnostic device 1 is provided with a configuration as a computer (computer).

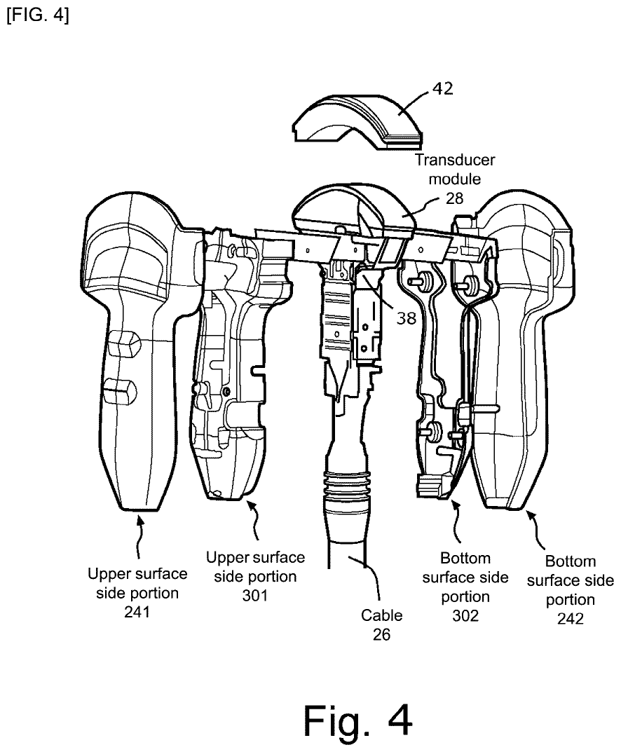

The ultrasonic probe 2 includes a plurality of ultrasonic transducers (see FIG. 4) disposed in an array, transmits ultrasonic waves to an examination target by the ultrasonic transducers, and receives an echo signal thereof.

The ultrasonic probe 2 transmits and receives ultrasonic waves to and from an examination target lying on a table (bed) 13.

The ultrasonic probe 2 includes a built-in magnetic sensor 12. The magnetic sensor 12 is also constituted by, for example, a Hall element, a magnetoresistive element, a magnetic impedance element, a GSR (GHz-Spin-Rotation) element, or a Faraday element. The magnetic sensor 12 detects the magnetism generated from the transmitter 11. The transmitter 11 and the magnetic sensor 12 are provided to detect a position and inclination of the ultrasonic probe 2 as will be described later.

Detection signals at the magnetic sensor 12 are input to the display processing unit 5. Detection signals at the magnetic sensor 12 may be input to the display processing unit 5 via a cable (see FIG. 4) or may be wirelessly input to the display processing unit 5.

The reception and transmission beamformer 3 supplies an electric signal for transmitting an ultrasonic wave from the ultrasonic probe 2 under a predetermined scanning condition to the ultrasonic probe 2 based on a control signal from the control unit 8. Furthermore, the reception and transmission beamformer 3 performs signal processing such as A/D conversion and delay-and-sum processing on the echo signals received by the ultrasonic probe 2 and outputs the signal-processed ultrasound data to the ultrasound data processing unit 4.

The ultrasound data processing unit 4 processes the ultrasound data output from the reception and transmission beamformer 3 to create an ultrasonic image. For example, ultrasound data processing unit 4 creates B-mode data by performing B-mode processing such as logarithmic compression processing or envelope detection processing.

The display processing unit 5 defines the position of the magnetic sensor 12 and the orientation of three axes orthogonal to each other set in the magnetic sensor 12 based on the magnetic detection signal from the magnetic sensor 12. The display processing unit 5 also defines a position and an orientation (orientation with respect to the three mutually orthogonal axes) of the magnetic sensor 12 in a coordinate system in three dimensional space with a transmitter 11 as the starting point.

The display processing unit 5 scan-converts data input from the ultrasound data processing unit 4 using a scan converter (scan converter) to create ultrasonic image data. For example, the display processing unit 5 scan-converts B-mode data to create B-mode image data and causes the display unit 6 to display an ultrasonic image based on the ultrasonic image data. The ultrasonic image is, for example, a B-mode image based on the B-mode image data.

In addition, the display processing unit 5 causes the display unit 6 to display, together with the ultrasonic image, a reference medical image of the same cross section as that of the ultrasonic image and the examination target. The data of the reference medical image is stored in a storage unit 9. The display processing unit 5 displays the ultrasonic image and the reference medical image of the same cross section of the examination target based on the position information of the echo signal and the coordinate conversion information defined by the coordinate conversion information. In certain embodiments, the reference medical image is a two dimensional image extracted from three dimensional image data from an X-ray CT or MRI device.

The display unit 6 is a liquid crystal display (LCD), an organic electro-luminescence (EL) display, or the like. The operation unit 7 is a device to which a user inputs instructions and information. For example, although not particularly illustrated in the drawings, the operation unit 7 includes a keyboard (keyboard), and also includes a pointing device (pointing device) such as a mouse (mouse), a trackball (trackball), and the like.

The control unit 8 is, for example, a processor such as a central processing unit (CPU). The control unit 8 reads a program stored in the storage unit 9 and controls each unit of the ultrasonic diagnostic device 1. For example, the control unit 8 reads a program stored in the storage unit 9 and causes the read program to execute the functions of the reception and transmission beamformer 3, the ultrasound data processing unit 4, and the display processing unit 5.

The control unit 8 may execute all of the functions of the reception and transmission beamformer 3, all of the functions of the ultrasound data processing unit 4, and all of the functions of the display processing unit 5 by a program, or may execute only a part of the functions by a program. When the control unit 8 executes only a part of the functions, the remaining functions may be executed by hardware such as a circuit. The functions of the reception and transmission beamformer 3, the ultrasound data processing unit 4, and the display processing unit 5 may be implemented by hardware such as a circuit.

The storage unit 9 is a hard disk drive (HDD), a semiconductor memory (memory) such as a random access memory (RAM) or a read only memory (ROM), or the like.

The ultrasonic diagnostic device 1 may include all of an HDD, a RAM, and a ROM as the storage unit 9. Furthermore, the storage unit 9 may be a portable storage medium such as a compact disk (CD) or a digital versatile disk (DVD).

A program executed by the control unit 8 is stored in a non-transient storage medium such as an HDD or a ROM. Furthermore, the program may be stored in a portable non-transient storage medium such as a CD or a DVD.

In addition to the above-described program, the storage unit 9 stores data of a reference medical image acquired in advance for the same examination target as the transmission/reception target of ultrasonic waves. The data of the reference medical image is data of a medical image acquired in advance by a medical image device other than the ultrasonic diagnostic device 1, that is, data of an X-ray CT image or data of an MRI image acquired in advance by, for example, an X-ray CT device or an MRI device. The data of the reference medical image is three dimensional data (volume data).

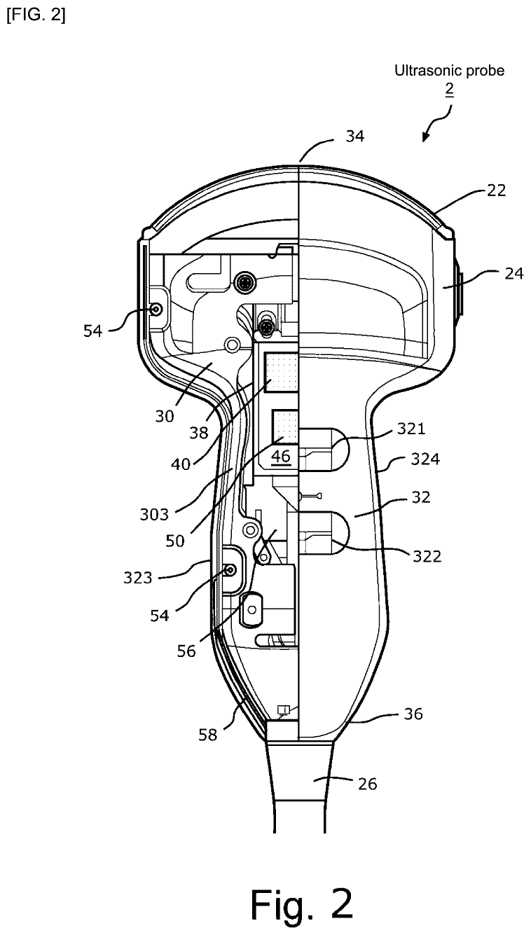

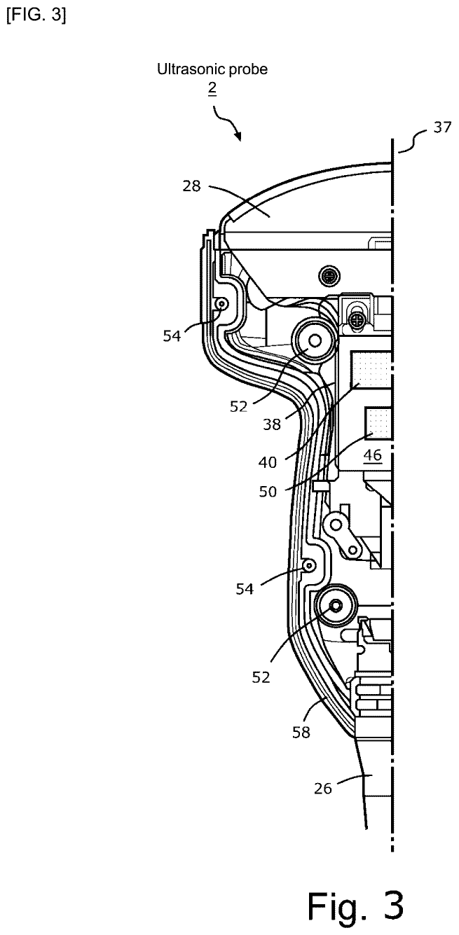

FIGS. 2 and 3 are diagrams illustrating an external structure and an internal structure of an ultrasonic probe 2. In the present embodiment, the ultrasonic probe 2 is a convex type ultrasonic probe, but the ultrasonic probe 2 may also be an ultrasonic probe for a bronchial endoscope, a transesophageal ultrasonic probe, or another type of ultrasonic probe such as a linear type or a sector type.

The right side of FIG. 2 is a diagram illustrating the external structure of the ultrasonic probe 2, and the left side of FIG. 2 is a diagram illustrating the internal structure of the ultrasonic probe 2 with the upper surface side portion 241 of the probe case 24 removed. As illustrated in FIG. 2, a lens 22 is disposed at the front end portion 34 of the ultrasonic probe 2, and the cable 26 is disposed at the back end portion 36 of the ultrasonic probe 2. An operator of the ultrasonic probe 2 holds a handle 32 and brings the lens 22 into contact with the examination target to collect ultrasonic images. The ultrasonic probe 2 has a rectangular cross-section in a plane perpendicular to a longitudinal axis 37 extending from the front end portion 34 to the back end portion 36.

In the embodiment of FIG. 2, as illustrated in FIG. 4, the probe case 24 of the ultrasonic probe 2 is constituted by an upper surface side portion 241 disposed on the front side of the paper surface of FIG. 2 and a bottom surface side portion 242 disposed on the back side of the paper surface of FIG. 2. The probe case 24 may be made of resin. As illustrated in FIG. 2, the bottom surface side portion 242 of the probe case 24 is provided with a plurality of protrusions 54 and the upper surface side portion 241 of the probe case 24 is provided with a plurality of holes that receive the plurality of protrusions 54 to enable precise alignment of the upper surface side portion 241 and the bottom surface side portion 242. Some or all of the plurality of protrusions 54 may be disposed on the upper surface side portion 241, and some or all of the plurality of holes that receive the protrusions 54 may be disposed on the bottom surface side portion 242. The bottom surface side portion 242 is further provided with a groove 58 at the end joined to the upper surface side portion 241 to receive a linear protrusion provided at the end of the upper surface side portion 241. The groove of the bottom surface side portion 242 and the linear protrusion of the upper surface side portion 241 may have complementary shapes that enable alignment with each other, and may be stepped portions or the like that are combined with each other.

A metal inner housing 30 is disposed inside the probe case 24 of the ultrasonic probe 2. The outer surface of the inner housing 30 has a shape conforming to the inner surface of the probe case 24. The inner housing 30 may be manufactured by a known method such as casting, additive manufacturing, CNC processing, forging, or press working. The inner surface of the probe case 24 is attached to the outer surface of the inner housing 30 by an adhesive. The upper surface side portion 241 and the bottom surface side portion 242 of the probe case 24 (FIG. 4) are also bonded to each other by an adhesive. The front end of the probe case 24 is adhered to the lens 22 and the back end of the probe case 24 is adhered to the cable 26. There are times when the ultrasonic probe 2 is sterilized with a sterilizing liquid and cleaned with a cleaning liquid, so the adhesive is preferably an adhesive having excellent water resistance, such as a polyvinyl chloride (PVC) resin-based adhesive or an epoxy resin-based adhesive. In terms of miniaturization, it is preferable that the thickness of the adhesive is 5 mm or less. In terms of the strength of the adhesive, it is preferable that the thickness of the adhesive is 0.3 mm or greater.

Returning to the description of FIG. 2, both an upper surface side portion 301 and a bottom surface side portion 302 of the inner housing 30 include an opening 56. The opening 56 is one aspect of a magnetic flux line-passing structure, and may be replaced with another structure that does not block the magnetic flux lines. Other structures that may be substituted include windows, slits, and non-magnetic materials. The inner housing 30 includes beam members 303 and 304 extending along sides 323 and 324 of the handle 32. The opening 56 of the inner housing 30 is at least partially defined by the beam members 303 and 304. The handle 32 is provided with one or more operation buttons 321 and 322, and the one or more operation buttons 321 and 322 are positioned at the position of the opening 56 of the inner housing 30. The positioning of the one or more operation buttons 321 and 322 at the position of the opening 56 of the inner housing 30 allows the one or more operation buttons 321 and 322 to sink slightly when an operator operates the one or more operation buttons 321 and 322.

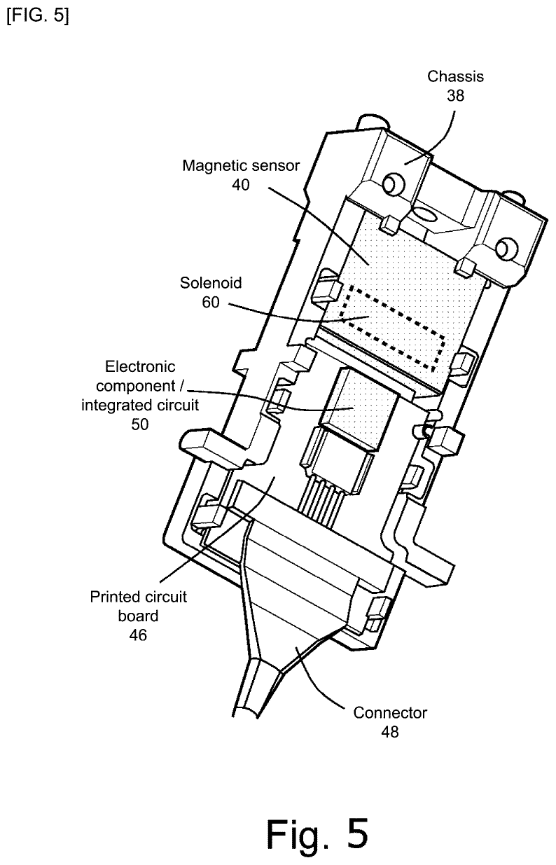

A chassis 38 is positioned in the opening 56 of the inner housing 30. A flexible printed circuit board 46 on which a magnetic sensor 40 and an electronic component 50 are disposed is fixed to the chassis 38. The chassis 38 is fixed to the inner housing 30 by a fastener such as a bolt so that constituent elements fixed thereto do not easily move and do not move from a predetermined position of the ultrasonic probe 2. A fastener such as a bolt for fixing the chassis 38 may be made of a non-magnetic material such as fine ceramic. The magnetic sensor 40 corresponds to the magnetic sensor 12 in FIG. 1. The electronic component 50 may be an integrated circuit that performs signal processing and processing related to environmental information. In certain embodiments of the present invention, the back end of the printed circuit board 46 is removably connected to the cable 26 by a connector 48 (FIG. 5) and the front end of the printed circuit board 46 is removably connected to a transducer module 28 by a connector (not illustrated). The chassis 38 is detachably fixed to the inner housing 30 by a fastener such as a bolt. When a failure or the like occurs in the magnetic sensor 40 and/or the electronic component 50, the chassis 38 including the printed circuit board 46 may be replaced with a new chassis through the opening 56 of the inner housing 30. In certain embodiments of the present invention, the chassis 38 is made of a non-magnetic material, such as a resin.

FIG. 3 is a diagram illustrating the internal structure of the ultrasonic probe 2 in which the upper surface side portion 301 (FIG. 4) of the inner housing 30 and a part of the lens 22 are removed. In the embodiment of FIG. 3, as illustrated in FIG. 4, the inner housing 30 of the ultrasonic probe 2 is constituted by the upper surface side portion 301 disposed on the front side of the paper surface of FIG. 3 and the bottom surface side portion 302 disposed on the back side of the paper surface of FIG. 3. As illustrated in FIG. 3, the bottom surface side portion 302 of the inner housing 30 is provided with a plurality of protrusions 52 and the upper surface side portion 301 of the inner housing 30 is provided with a plurality of holes that receive the plurality of protrusions 52 to enable precise alignment of the upper surface side portion 301 and the bottom surface side portion 302. Some or all of the plurality of protrusions 52 may be disposed on the upper surface side portion 301, and some or all of the plurality of holes that receive the protrusions 52 may be disposed on the bottom surface side portion 302. The bottom surface side portion 302 may be further provided with a groove at the end joined to the upper surface side portion 301 to receive a linear protrusion provided at the end of the upper surface side portion 301. The groove of the bottom surface side portion 302 and the linear protrusion of the upper surface side portion 301 may have complementary shapes that enable alignment with each other, and may be stepped portions or the like that are combined with each other.

The plurality of protrusions 52 of the inner housing 30 may be replaced by fasteners such as bolts and nuts. The upper surface side portion 301 and the bottom surface side portion 302 of the inner housing 30 are also bonded to each other by an adhesive. The front end of the inner housing 30 is adhered to the transducer module 28, and the back end of the inner housing 30 is adhered to the cable 26. There are times when the ultrasonic probe 2 is sterilized with a sterilizing liquid and cleaned with a cleaning liquid, so the adhesive is preferably an adhesive having excellent water resistance, such as a polyvinyl chloride (PVC) resin-based adhesive or an epoxy resin-based adhesive. The upper surface side portion 301 and the bottom surface side portion 302 of the inner housing 30 may be joined by another method such as welding. While the inner housing 30 is a rigid body made of metal, the cable 26 is made of resin and has flexibility, and thus becomes a portion into which liquid enters in a relatively easy manner. In certain embodiments, the cable 26 and the inner housing 30 are provided with one or more annular grooves and one or more annular protrusions that are complementary to each other to prevent liquid from entering the interior of the ultrasonic probe 2. One or more rubber O-rings may be disposed in the one or more annular grooves to enhance air and water tightness.

FIG. 4 is an exploded view illustrating main components of the ultrasonic probe 2, and FIG. 5 is a diagram illustrating the chassis 38 that is built into the ultrasonic probe 2. As illustrated in FIG. 5, the printed circuit board 46 of the chassis 38 is connected at the back end thereof to the connector 48 of the cable 26. The printed circuit board 46 of the chassis 38 is connected to the transducer module 28 at the front end of the chassis 38. An inner lens 44, followed by a hard shell lens 42, are attached to the transducer module 28. Next, the upper surface side portion 301 and the bottom surface side portion 302 of the inner housing 30 are also bonded to each other so as to enclose or interpose these components. Next, the upper surface side portion 241 and the bottom surface side portion 242 of the probe case 24 are joined to each other so as to enclose or interpose these components. The assembly method described in the embodiment of FIG. 4 may be carried out in various ways. For example, it is also possible to manufacture as a single structure by additive manufacturing or casting without the inner housing 30 being divided into the upper surface side portion 301 and the bottom surface side portion 302. The inner housing 30 may also be formed by joining components divided in the longitudinal direction or the latitudinal direction. The probe case 24 may also be formed by joining components divided in the longitudinal direction or the latitudinal direction.

As illustrated in FIG. 5, in certain embodiments of the present invention, the magnetic sensor 40 is disposed on the front end side of the printed circuit board 46 of the chassis 38, and the electronic component 50 is disposed on the back end side thereof. In certain embodiments of the present invention, the magnetic sensor 40 has a built-in solenoid 60.

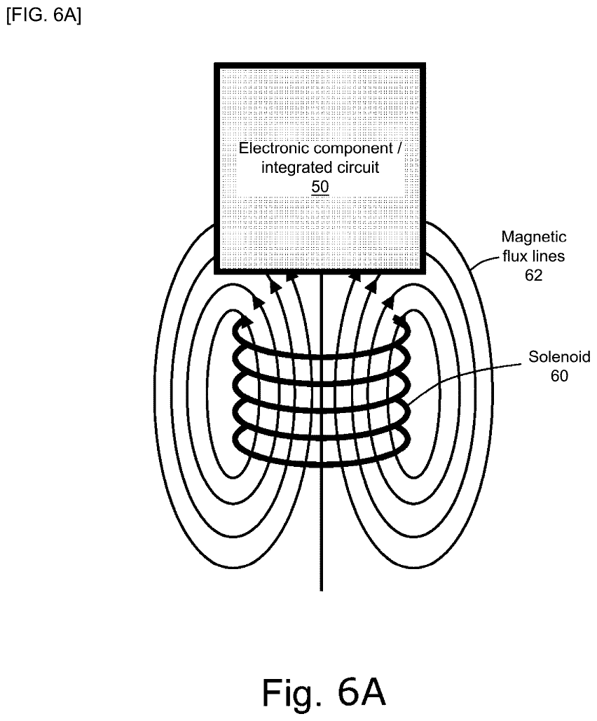

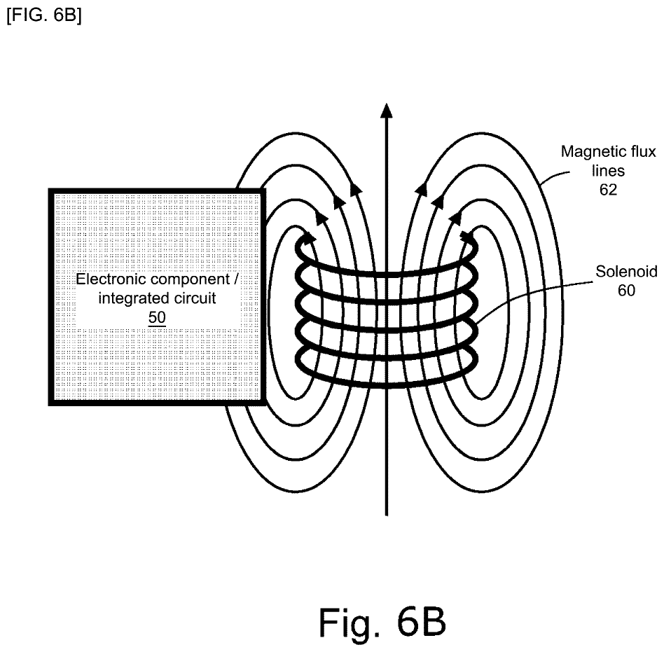

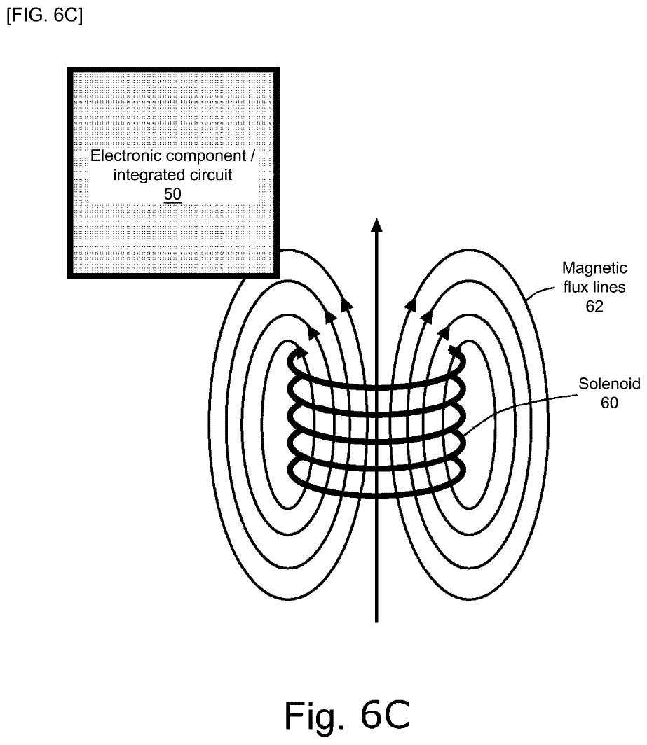

FIG. 6A to FIG. 6C are diagrams illustrating the disposal of the solenoid 60 of the magnetic sensor 40 built into the ultrasonic probe 2 and the integrated circuit (electronic component) 50 built into the ultrasonic probe 2. In the present embodiment, the magnetic sensor 40 supplies power from the cable 26 to the solenoid 60 to generate a plurality of magnetic flux lines 62 from the solenoid 60. The solenoid 60 is an example of a magnetic flux line generator having a first pole and a second pole. The magnetic sensor 40 determines the position and orientation of the solenoid 60 of the magnetic sensor 40 using changes in the signals of the magnetic flux lines due to the influence of the magnetic field of the transmitter 11.

FIG. 6A illustrates an example wherein the electronic component 50 is disposed above the N pole of the solenoid 60, FIG. 6B illustrates an example wherein the electronic component 50 is disposed on the side of the solenoid 60, and 6C illustrates an example wherein the electronic component 50 is disposed obliquely above the N pole of the solenoid 60.

The inventors of the present application confirmed that the magnetic flux lines of the solenoid 12 of the magnetic sensor 40 do not adversely affect the signal processing of the shielded electronic component 50, and the presence of the electronic component 50 also does not adversely affect the signals of the magnetic flux lines of the magnetic sensor 40 in any of the examples in FIG. 6A to FIG. 6C. The inventors of the present application confirmed that the electronic component 50 can be disposed in any orientation with respect to the solenoid 60, such as vertically, horizontally, or in a twisted manner.

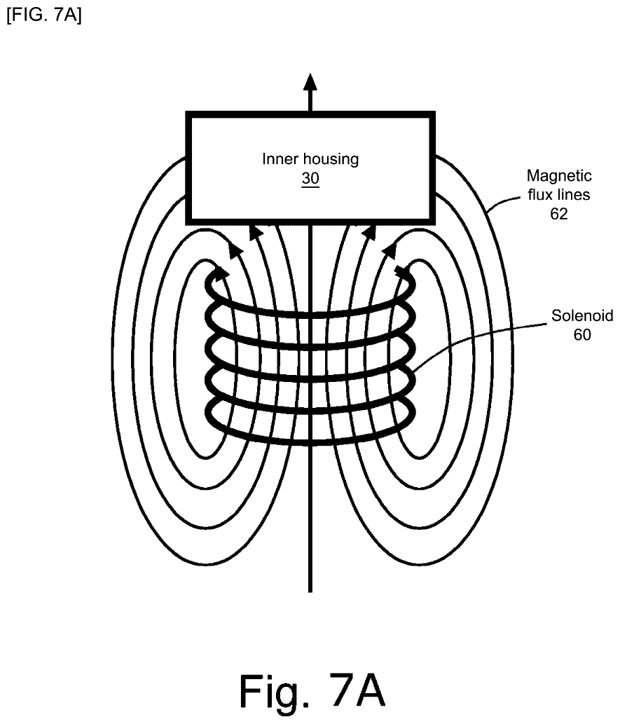

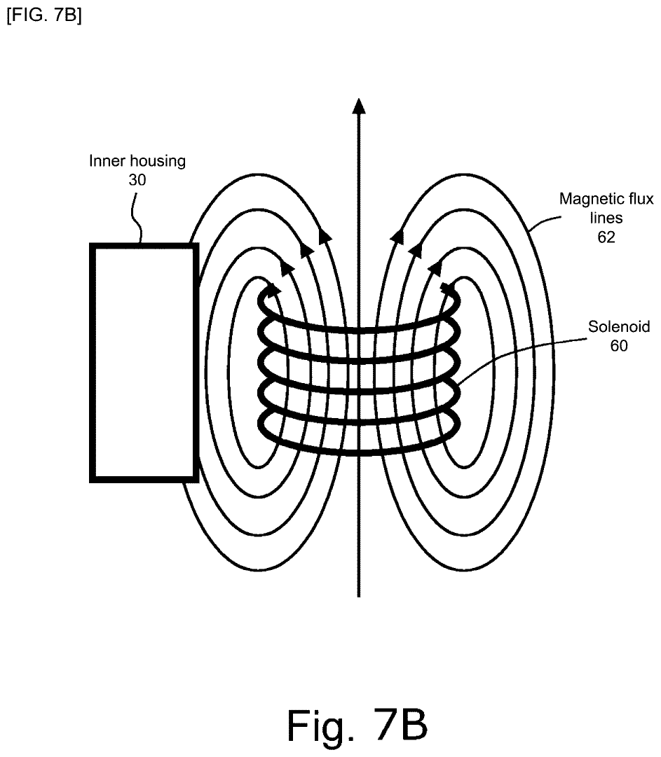

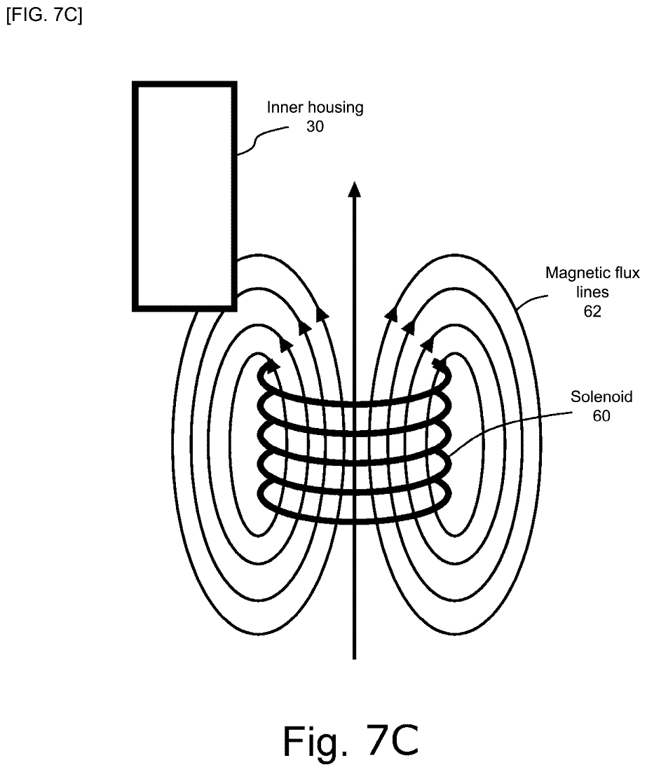

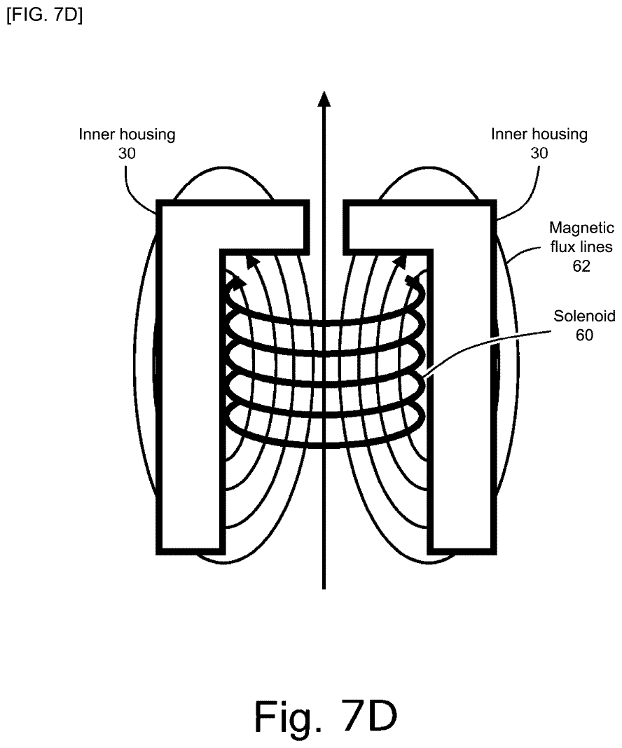

FIG. 7A to FIG. 7D are diagrams illustrating the disposal of the solenoid 60 of the magnetic sensor 40 built into the ultrasonic probe 2 and the metal member of the inner housing 30 built into the ultrasonic probe 2. In the present embodiment, the magnetic sensor 40 generates a plurality of magnetic flux lines 62 from the solenoid 60, and the magnetic sensor 40 determines the position and orientation of the solenoid 60 of the magnetic sensor 40 from changes in the signals of the magnetic flux lines.

As described above, the ultrasonic transducer included in the ultrasonic probe vibrates in response to an applied voltage and generates heat because it is a constituent element that generates ultrasonic waves. In order to dissipate heat generated by the ultrasonic transducer, the ultrasonic probe may be provided with a metal inner housing 30 that is thermally connected to the ultrasonic transducer. Since the inner housing 30 must have high thermal conductivity, it must be manufactured using a metal having high thermal conductivity, such as aluminum or copper. In terms of weight reduction, aluminum is more preferable than copper. In addition, when the inner housing 30 is thin, heat accumulation around the ultrasonic transducer is prevented and heat transfer capability is lowered, so the thickness must be 1 mm or greater. On the other hand, when the inner housing 30 is too thick, the heat transfer capability improves, but the ease of processing is reduced and the weight of the ultrasonic probe cannot be reduced, so the thickness must be 5 mm or less. More preferably, the thickness of the inner housing 30 is 2 to 4 mm.

FIG. 7A illustrates an example wherein the metal of the inner housing 30 is disposed above the N pole of the solenoid 60, FIG. 7B illustrates an example wherein the metal of the inner housing 30 is disposed on the side of the solenoid 60, FIG. 7C illustrates an example wherein the metal of the inner housing 30 is disposed obliquely above the N pole of the solenoid 60, and FIG. 7D illustrates an example wherein the upper side of the N pole of the solenoid 60 is slightly opened and the upper side and the side of the N pole of the solenoid 60 are disposed so as to surround the metal of the inner housing 30.

The inventors of the present invention have found that the magnetic sensor 40 does not function when the metal material of the inner housing 30 blocks many of the loops of the magnetic flux lines as illustrated in FIG. 7A and FIG. 7D, whereas the magnetic sensor 40 performs sufficiently (or to a minimum degree) when the metal material of the inner housing 30 does not block many of the loops of the magnetic flux lines as illustrated in FIG. 7B and FIG. 7C.

That is, as illustrated in FIG. 7A, when metal is disposed at a position orthogonal to the direction of magnetic flux, all magnetic flux generated from the solenoid passes through the metal and cannot be detected as a signal. When metal is disposed in a parallel position as illustrated in FIG. 7B, a part of the magnetic flux that is generated passes through the metal, and the remaining magnetic flux can be used as a signal (magnetic flux on the right side of in FIG. 7B). As in FIG. 7C, the magnetic sensor 40 can function because disposing between an orthogonal and a parallel position makes it possible for only a portion of magnetic flux to penetrate the metal as in the FIG. 7B. However, when the disposing arrangements in FIG. 7B and FIG. 7C are combined as in FIG. 7D, the amount of magnetic flux that does not pass through the metal becomes small, and thus the signal strength decreases. That is, the magnitude of the magnetic flux density passing through the metal greatly affects the signal strength.

When the metal of the inner housing 30 blocks many of the loops of magnetic flux lines as illustrated in FIG. 7D, the magnetic flux lines generate eddy currents in the metal of the inner housing 30. That is, the magnetic energy is converted into electrical energy and the magnetic energy is lost. As a result, the magnetic sensor 40 cannot detect changes in the signals of magnetic flux lines, and the functionality of the magnetic sensor 40 is lost. Eddy currents may generate magnetic forces and cause noise. Although not easy to quantitatively generalize because it is determined by the transmission intensity of magnetic flux emitted from the solenoid 60 and the sensitivity of the receiving circuit, adopting a design in which the metal is disposed so as not to pass through the magnetic flux 62 emitted from the solenoid 60 and confirming the function of the magnetic sensor 40 makes it possible to determine a disposal of metal that does not significantly reduce the functionality of the magnetic sensor 40.

In addition to the magnitude of the flux density passing through the metal, the size and shape of the metal 30 also affects the signal strength of the magnetic sensor 40. This is because when the magnetic sensor 40 moves while magnetic flux passes through the metal 30, an eddy current 72 is generated in the metal 30, and an induced magnetic field 70 is generated by electromagnetic induction from the eddy current 72 to cause signal interference. As the resistance of the metal decreases and the size of the metal increases, the generated eddy current 72 increases and the strength of the interfering magnetic field 70 increases. The inner housing 30 must have a high thermal conductivity, and a metal having a high thermal conductivity generally has a low electrical resistance. In order to avoid the eddy current 72, the metal 30 is required to have a certain volume or less. On the other hand, the metal 30 is required to have a large volume in order to achieve heat diffusion. In order to solve the problem of the eddy current 72, it is effective to form a slit or a window in the metal of the inner housing 30 to reduce the loops of the eddy current 72. The slit or the window reduces the loop area of the eddy current, reduces the magnetic flux of the induced magnetic field 70 generated based on Faraday's law, and reduces the interference signal. Here, a slit refers to an open loop starting from an end face of the metal and ending at another end face or in the metal, and a window refers to a closed loop starting from in the metal and ending in the metal. Unlike an opening provided for fastening or the like, the window or the slit provided in the inner housing 30 is maintained in a state where the fastener is not inserted even when the ultrasonic probe 2 is operated. The window or slit may be reinforced by filling it with a non-magnetic material. It is also possible to increase the thermal conductivity of the inner housing 30 by filling the inner housing 30 with a nonmagnetic material having high thermal conductivity such as a thermally conductive nylon resin. Using a resin having high thermal conductivity improves the rigidity and thermal conductivity of the inner housing 30, so the area and/or volume of the magnetic flux line-passing structure can be increased. While the thermal conductivity of general nylon is about 0.2 W/m·K, high thermal conductive resins have a thermal conductivity of 1.0 W/m·K or greater. Further, on portions of the inner housing 30 where it is desirable to prevent eddy current from occurring, for example, silicon may be added to the metal of the inner housing 30 to increase the electric resistance, or a laminated structure may be partially introduced to reduce eddy current.

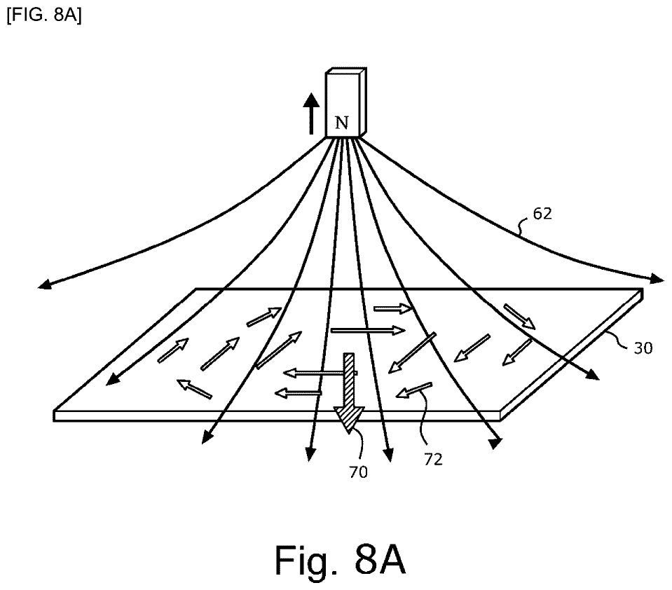

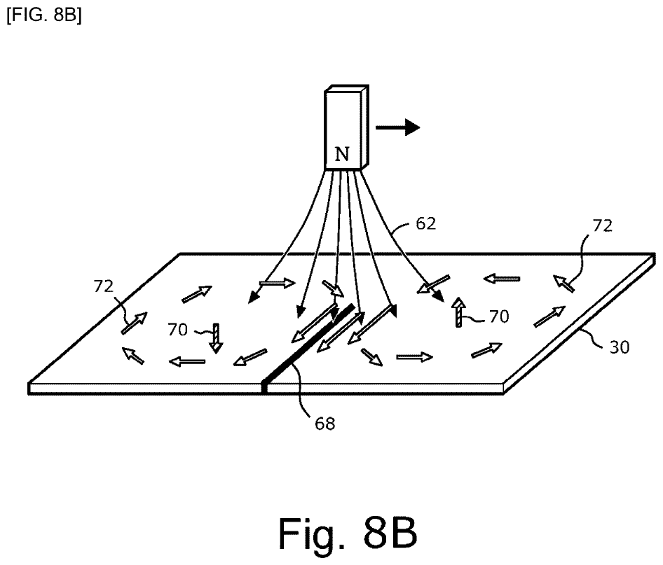

FIG. 8A illustrates eddy currents 72 generated in the metal 30 that does not have the slit 68, and FIG. 8B illustrates eddy currents 72 generated in metal 30 that has the slit 68. When the slit 68 is provided, as illustrated in FIG. 8B, eddy currents 72 having the large loop generated in FIG. 8A do not occur, but eddy currents 72 having a small loop divided by the slit 68 do occur. As described above, it is possible to provide a qualitative design rule that when the metal 30 has a certain volume or greater, the slit 68 is formed so as not to generate a large eddy current 72. However, since an allowable metal size or the like is determined by the magnetic flux 62 generated from the solenoid 60 and the sensitivity of the receiving circuit, it is not necessarily easy to quantitatively generalize such a design rule.

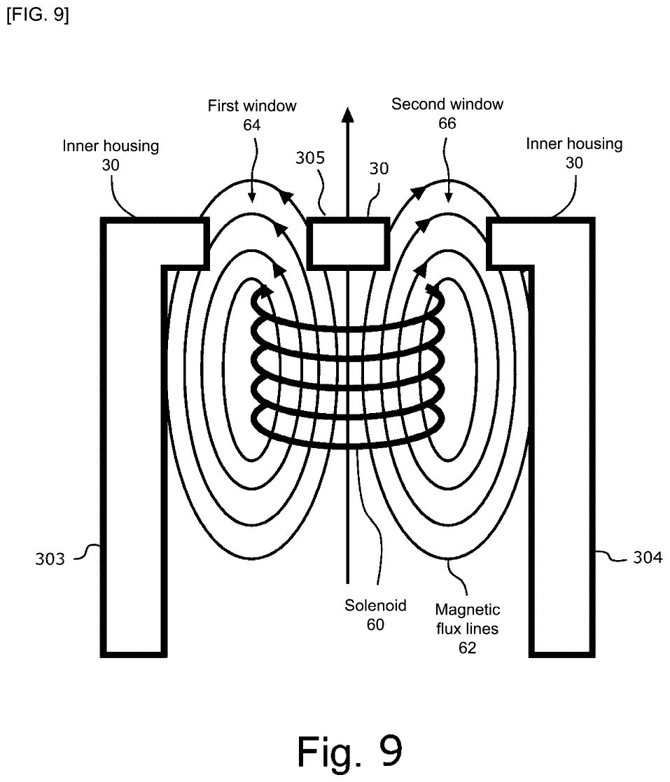

FIG. 9 is a conceptual diagram illustrating an example of the inner housing 30 that does not significantly affect the magnetic flux lines 62 of the solenoid 60. As illustrated in the drawings, the N pole of the solenoid 60 is disposed at the top (in a direction toward the front end portion 34 of the ultrasonic probe 2). The N pole of the solenoid 60 may be disposed downward (in a direction toward the back end portion 36 of the ultrasonic probe 2). However, when the N pole of the solenoid 60 is disposed in the lateral direction, the magnetic flux lines 62 are blocked by the beam members 303 and 304 of the inner housing 30, which is not preferable. Since the beam members 303 and 304 of the inner housing 30 extend along both side portions 323 and 324 of the ultrasonic probe 2, the operator is positioned at the handle 32 holding the ultrasonic probe 2, and the beam members 303 and 304 need to bear the rigidity of the ultrasonic probe 2, it is not easy to provide the slit 68 in the beam members 303 and 304.

The inner housing 30 must be in contact with the ultrasonic transducer 28 disposed at the front end portion 34 of the ultrasonic probe 2, which is a heat source, as uniformly as possible to diffuse heat to the back end portion 36 side. For this reason, the front end portion 305 of the inner housing 30 preferably extends over the entire width of the ultrasonic probe 2. In order to simultaneously satisfy the requirement of heat conduction, the requirement of not blocking magnetic flux lines, and the requirement of not generating large loop eddy currents, the inner housing 30 in FIG. 9 is provided with a first window 64 and a second window 66 at the front end portion 305 to allow magnetic flux lines to pass through. The first window 64 and the second window 66 may be replaced by other magnetic flux line-passing structures, by disposing a non-metallic material or the like. The first window 64 and the second window 66 are given a position and size to allow a detectable amount of magnetic flux to pass through the magnetic field of the transmitter 11.

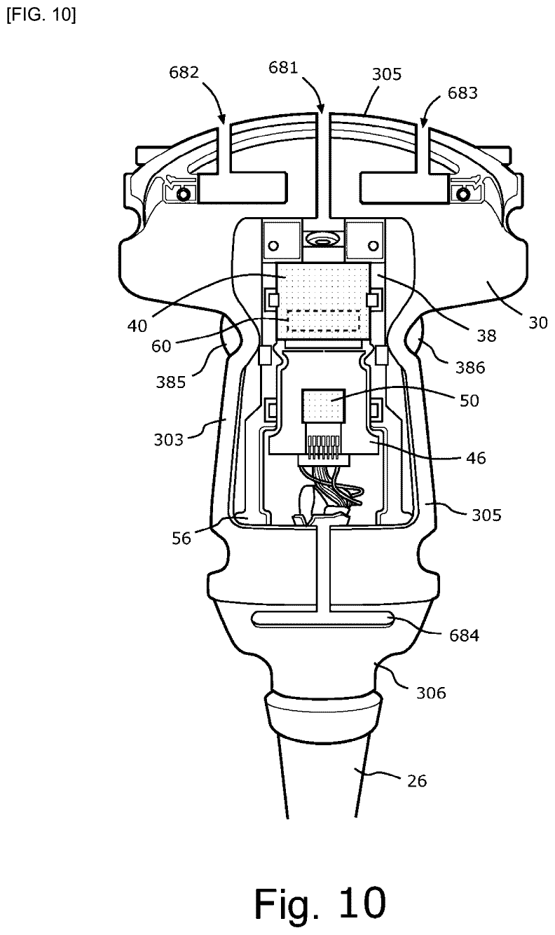

FIG. 10 is a diagram illustrating the shape of the inner housing 30 and the positions at which the magnetic sensor 40 and the electronic component 50 are disposed in the inner housing 30. In FIG. 10, the inner housing 30 is connected to the cable 26 and has a built-in chassis 38. A printed circuit board 46 on which a magnetic sensor 40 and an electronic component 50 are disposed is fixed to the chassis 38. The chassis 38 also limits movement of the magnetic sensor 40 using electromagnetic induction. The chassis 38 is accessible through the opening 56 of the inner housing 30 to facilitate assembly of the ultrasonic probe 2 and replacement of the chassis 38.

In the example of FIG. 10, the front end portion 305 of the inner housing 30 is provided with a slit 681 extending from the end of the front end portion 305 to the opening 56 at the center of the front end portion 305 of the inner housing 30 and completely dividing the front end portion 305 of the inner housing 30 into left and right portions, and T-shaped slits 682 and 683 disposed on the left and right sides thereof. The slit 681 extends along the longitudinal axis 37. In order to make the thermal distribution generated in the transducer module 28 uniform and promote thermal conduction to the back end portion 305, the width of the slit 681 is set to 1 to 5 mm, preferably 2 to 4 mm, and more preferably 2.5 to 3.5 mm. The widths of the other slits 682, 683, and 684 illustrated in FIG. 10 are set in the same manner. The back end portion 306 of the inner housing 30 is provided with a T-shaped slit 684 extending from the opening 56 toward the cable 26. A left portion of the front end portion 305 and a right portion of the front end portion 305 are supported by the back end portion 306 which is not completely divided. A window (not illustrated) may also be provided in each of the left portion of the front end portion 305 and the right portion of the front end portion 305. The left beam member 303 may be provided with a window 385, and the right side beam member 304 may be provided with a window 386.

Slits and windows having exactly the same positions and shapes as those of the upper surface side portion 301 of the inner housing 30 may also be disposed on the bottom surface side portion (bottom surface side half) 302 of the inner housing 30 disposed on the back side of the paper surface of FIG. 10. Since the inner housing 30 occupies a large proportion of the total weight of the ultrasonic probe 2, it is possible to provide a weight balance that is easy for the operator to operate by making the slit and the window bilaterally symmetrical and making the top surface and the bottom surface symmetrical. That is, the ultrasonic probe 2 may be bilaterally symmetrical in terms of shape and weight, and may be symmetrical on the top surface and the bottom surface. Furthermore, by adjusting the size and the number of slits and windows provided in the front end portion 305 and the back end portion 306 of the inner housing 30, it is possible to adjust the weight balance between the front and the back of the ultrasonic probe 2. In the example of FIG. 10, the front end portion 34 of the ultrasonic probe 2 is heavier than the back end portion 36. This weight balance makes it easier for the ultrasonic probe 2 to be immersed in an antiseptic solution or a cleaning solution with the front end portion 34 of the ultrasonic probe 2 facing downward.

Due to the shape of the inner housing 30 illustrated in FIG. 10 and the disposal of the magnetic sensor 40 and the electronic component 50, the inner housing 30 performs the required heat dissipation function (ensures continuous operation), the magnetic sensor 40 performs the function of detecting the position and orientation of the ultrasonic probe 2, and the electronic component 50 can perform the required signal processing and environmental information acquisition function without signal interference caused by the induced magnetic field 70 generated by electromagnetic induction from the eddy current 72 generated in the inner housing 30.

In the above-described embodiment, the magnetic sensor 40 supplies power from the cable 26 to the solenoid 60 to generate a plurality of magnetic flux lines 62 from the solenoid 60 and determines the position and orientation of the solenoid 60 of the magnetic sensor 40 based on changes in the signals of the magnetic flux lines due to the influence of the magnetic field of the transmitter 11. However, the present invention can also be applied to a magnetic sensor such as a Hall element type, a magnetoresistive element type, a magnetic impedance element type, a GHz-Spin-Rotation (GSR) element type, or a Faraday element type as the magnetic sensor 40. That is, a magnetic flux line-passing structure such as a slit, a window, or a non-magnetic material provided in the inner housing 30 allows the magnetic flux line of the transmitter 11 outside the ultrasonic probe 2 to reach the inside of the inner housing 30, and the magnetic sensor 40 disposed inside the inner housing 30 can detect the magnetic flux lines from the transmitter 11.

Although a description was given in the embodiment above in which the magnetic sensor 40 is disposed inside the ultrasonic probe 2, instead of this embodiment, the position and orientation of the ultrasonic probe 2 may be defined by disposing the transmitter 11 inside the ultrasonic probe 2 and detecting a magnetic field from the inside of the ultrasonic probe 2 by one or more magnetic sensors disposed outside the ultrasonic probe 2.

Furthermore, constituent elements related to the magnetic force may be disposed inside the ultrasonic probe 2 independently of the detection of the position and/or orientation of the ultrasonic probe 2. In other words, a magnetic field generating element and/or a magnetic field receiving element may be present in the ultrasonic probe 2. Regarding the magnetic field generating element, for example, an electromagnet may be disposed inside the ultrasonic probe 2, and an image of the inside of the imaging target may be acquired by turning on the electromagnet and attracting a movable magnetic body inside the imaging target by magnetic force, or an image of the inside of the imaging target may be acquired by turning off the electromagnet and not attracting the movable magnetic body inside the imaging target. Even in such examples, the magnetic flux line-passing structure provided in the inner housing 30 functions effectively.

Note that the invention is not limited to the present embodiment, and various modifications are possible without departing from the gist of the invention.

### DESCRIPTION OF CODES

1 Ultrasonic diagnostic device
2: Ultrasonic probe
3: Reception and transmission beamformer
4: Ultrasound data processing unit
5: Display processing unit
6: Display unit
7: Operation unit
8: Control unit
9: Storage unit
11: Transmitter
12: Magnetic sensor
13: Table
22: Lens
24: Probe case
241: Upper surface side portion
242: Bottom surface side portion
26: Cable
28: Transducer module
30: Inner housing
301: Upper surface side portion
302: Bottom surface side portion
303, 304: Beam member
305: Front end portion
306: Back end portion
32: Handle
321, 322: Operation button
323, 324: Side portion
34: Front end portion
36: Back end portion
37: Longitudinal axis
38: Chassis
40: Magnetic sensor
42: Hard shell lens
44: Inner lens
46: Printed circuit board
48: Connector
50: Electronic component/integrated circuit
52: Protrusion
54: Protrusion
56: Opening
58: Groove
60: Solenoid
62: Magnetic flux line
64: First window
66: Second window
68: Slit
681, 682, 683, 684: Slit
685, 686: Window
70: Induced magnetic field
72: Eddy current
100: Ultrasonic diagnostic system

What is claimed is:

1. An ultrasonic probe, comprising:
an ultrasonic transducer;
a metal inner housing that is thermally connected to the ultrasonic transducer; and
a magnetic flux line generator disposed at a position at least partially surrounded by the inner housing and having a first pole and a second pole, wherein
the inner housing comprises a magnetic flux line-passing structure that allows passage of magnetic flux lines from the first pole back to the second pole.

2. The ultrasonic probe of claim 1, wherein the magnetic flux line generator is included in a magnetic sensor that detects a magnetic field of a transmitter disposed outside the ultrasonic probe, and the magnetic flux line-passing structure has a position and size that allows passage of a detectable amount of magnetic flux through the magnetic field of the transmitter.

3. The ultrasonic probe according to claim 2, wherein the magnetic flux line-passing structure includes any one or more of a slit, a window, and a non-metal material.

4. The ultrasonic probe according to claim 2, wherein the magnetic sensor detects a position and/or an orientation of the ultrasonic probe.

5. The ultrasonic probe according to claim 2, wherein the magnetic flux line generator includes a solenoid that is supplied with electric power to generate a magnetic force.

6. The ultrasonic probe according to claim 5, comprising a cable disposed at the back end portion of the ultrasonic probe and supplying the electric power to the solenoid.

7. The ultrasonic probe according to claim 2, further comprising a chassis that fixes the magnetic sensor at a predetermined position in the inner housing, wherein the chassis is formed of a resin material.

8. The ultrasonic probe according to claim 2, further comprising a probe case that houses the inner housing therein.

9. The ultrasonic probe according to claim 8, wherein the probe case is adhered to the inner housing by an adhesive.

10. The ultrasonic probe according to claim 8, wherein:
the probe case includes a top surface, a bottom surface, and first and second side surfaces connected to the top surface and the bottom surface;
the inner housing (30) includes a first beam member (303) extending along a first side surface of the probe case and a second beam member (304) extending along a second side surface of the probe case;
the magnetic sensor is disposed between the first beam member and the second beam member; and
the first pole and the second pole are disposed along a longitudinal axis (37) extending from a front end portion of the ultrasonic probe to a back end portion of the ultrasonic probe.

11. The ultrasonic probe according to claim 10, comprising at least one operation button on the upper surface of the probe case, wherein the at least one operation button is positioned between the first beam member and the second beam member.

12. The ultrasonic probe according to claim 8, wherein the shape of an outer surface of the inner housing is configured to correspond to the shape of an inner surface of the probe case, and wherein a gap formed between the probe case and the inner housing is 5 mm or less.

13. The ultrasonic probe according to claim 10, wherein the probe case is made of resin, and the inner housing provides main rigidity of the ultrasonic probe.

14. The ultrasonic probe according to claim 13, wherein the ultrasonic probe has a rectangular cross-section in a plane perpendicular to a longitudinal axis (37) extending from the front end portion to the back end portion.

15. The ultrasonic probe according to claim 2, wherein:
the ultrasonic transducer is disposed at a front end portion of the ultrasonic probe;
the inner housing extends from the front end portion to a back end portion of the ultrasonic probe;
the front end portion of the inner housing has a first front end portion and a second front end portion that are completely separated by a first slit; and
the first front end portion and the second front end portion are supported by a back end portion of the inner housing that is not completely divided.

16. The ultrasonic probe according to claim 15, wherein the first front end portion and/or the second front end portion comprises one or more windows.

17. The ultrasonic probe according to claim 2, wherein the inner housing has a thickness of 1 to 5 mm.

18. An ultrasonic probe, comprising:
an ultrasonic transducer;
a metal inner housing that is thermally connected to the ultrasonic transducer; and
a magnetic sensor disposed at a position at least partially surrounded by the inner housing, wherein:
the inner housing comprises a magnetic flux line-passing structure that allows passage of magnetic flux lines to detect a magnetic field generated by a transmitter disposed outside the ultrasonic probe.
